(19) United States
(12) Patent Application Publication
(10) Pub. No.: US 2023/0157779 A1
(43) Pub. Date: May 25, 2023

(54) PIVOTING SURGICAL IMPLANT PLACEMENT TOOL

(71) Applicant: Neuralink Corp., Fremont, CA (US)

(72) Inventors: Russell N. Ohnemus, San Francisco, CA (US); Robin E. Young, San Francisco, CA (US)

(73) Assignee: Neuralink Corp., Fremont, CA (US)

(21) Appl. No.: 17/531,244

(22) Filed: Nov. 19, 2021

(51) Int. Cl. A61B 34/00; A61B 34/30; A61B 17/34

(52) U.S. Cl. CPC A61B 34/71; A61B 34/30; A61B 17/3468; A61B 2034/305; A61B 2017/00743

(57) ABSTRACT

Disclosed is a brain implant placement apparatus, which includes a base member contoured to rest against a cranium of a patient. The brain implant apparatus including a pivot arm hingedly coupled to the base member and configured to rotate from a first configuration to a second configuration. The brain implant placement apparatus including an implant attachment member coupled to the pivot arm via a rotating attachment mechanism and further comprising a receiving portion configured to removably couple with a brain implant and rotate the brain implant. In the first configuration, the pivot arm is positioned such that the receiving portion of the implant attachment member is facing upward. In the second configuration, the pivot arm is positioned such that the receiving portion of the implant attachment member is facing substantially downwards.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH AND DEVELOPMENT

NOT APPLICABLE

### BACKGROUND

A fundamental technical barrier to understanding and repairing the brain (and brain function) is the limited availability to develop data related to neural activity. Often, information received from monitoring a single neuron in isolation is not helpful to understanding brain activity overall. Thus, neural implant operations may include monitoring a plurality of neurons throughout the brain. Accessing data from multiple electrodes, and thus, multiple neurons, typically requires support circuitry connecting with a central unit. Thus, communication with the brain may be provided by support circuitry, such as wires in ribbon cables, that allow for receiving electrode signals to a central unit (the brain implant device) within the cranium. These electrodes are typically fragile, due to their desirable small sizing, which allows for receiving and sending signals to an individual neuron.

Support circuitry is often desirable for increased sensor accuracy to specifically identify which individual electrodes are responding to a neuron in a group of electrodes. Support circuitry (like the electrodes themselves) is also often fragile, and once implantation is complete, is not easily reparable. Given the size, fragility and complexity of such electrodes and support circuitry, precision and delicate control are necessary to ensure electrodes are properly and individually attached to their respective neurons. To help ensure this, alignment and stability of the central unit during the implantation process are both important factors to ensure no pinching, crimping, or damage is caused to the support circuitry and electrodes, which may otherwise require subsequent procedures and intervention.

Current methods for electrode implantation rely heavily on the capabilities of the physician or technician performing the procedure. While such technicians may be assisted by robotic or animated tools, human intervention is generally required for many steps which are not performed by an animated or robotic device, such as positioning of the implant device. As such, current brain implant procedures rely heavily and often hinge on the qualifications and skills of the physician or technician performing the procedure. While a fully robotic or animated procedure may not be possible or suitable in many situations, devices and tools which limit or assist in limiting the complexity of human intervention by the physician or technician during the positioning and alignment of the brain implant device may prevent or reduce potentials for error or failure of the procedure.

Therefore, there is a need in the art for an improved mechanism of brain implantations which allows for greater control and stability without risking damage to electrodes and accompanying support circuitry.

### BRIEF SUMMARY

Implementations of the present disclosure are related to a brain implantation tool having a base member that fits to the cranium of an implantation subject (patient), with an apparatus for stabilizing and controlling implantation, and methods of use for the same.

In various implementations, the present disclosure is related to a brain implant apparatus, designed to allow ease of maneuverability and positioning of an implant, with a compact design to allow access and visibility to the implant placement site. More specifically, the brain implant apparatus according to the implementations discussed herein allows the implant to be rotated or pivoted in use, such that the implant may be initially inverted or partially inverted to allow ease of access to the embeddable portion of the implant during installation of the electrodes into the implant placement site.

The brain implant (also “implant”) is described herein as being of cylindrical or mostly cylindrical shape configured to be inserted into a round opening in the cranium of a patient, the implant placement site. As described, the implant has an embeddable portion, configured to be inserted into the cranium of a patient, a top portion opposite the embeddable portion, and a cylindrical side portion. While described as being cylindrical herein, it should be understood that various implementations may be of cubic, rectangular, oval, or other suitable shape, as is understood by one skilled in the art. The implant may further include mounting eyelets protruding from the sides of the implant, which may be configured to receive screws for securing the implant to the exterior surface of the cranium of the patient.

In one aspect, the brain implant apparatus may include a base member dimensioned and configured to be positioned on the cranium of a patient. The base member may include one or more attachment holes which may be sized to receive fastening screws (“apparatus screws”). In various implementations, such fastening may include use of biocompatible materials and devices, including biocompatible screws. The base member may be of suitable shape to partially or completely surround the implant placement site. For example, the base member may be U-shaped, rectangular, semi-circular, or circular.

In another aspect, the brain implant apparatus may include a pivot arm which hingedly couples to the base member. The pivot arm may attach to the base member via one, two, or more hinge locations. In some implementations, the hinge may operate by use of screws or bolts which connect the base member to the pivot arm. The pivot arm may be configured to allow the implant to pivot by 180 degrees or more, or, in various implementations, to pivot between 90 and 180 degrees. For example, in use, the pivot arm may be positioned in a first configuration such that the embeddable portion of the implant is facing upward or mostly upward (away from the cranium of the patient). The pivot arm may then be pivoted to a second configuration, where the embeddable portion of the implant is facing substantially downward (towards the cranium of the patient). In some implementations, the second configuration allows the implant to be positioned concentrically with the implant placement site, such as directly aligned with and above the implant placement site.

The pivot arm may further include a locking screw, wherein the locking screw fastens the pivot arm to the base member, preventing rotation of the pivot arm. For example, in use, the locking screw, when fastened, prevents the pivot arm from rotating from the first position to the second position. While described as a locking screw herein, other means of preventing movement of the pivot arm are contemplated, including other fasteners, clips, latches, or locks. Further, in various implementations the locking screw may be positioned on the base member and function similarly to the locking screw previously described.

In another aspect, the brain implant apparatus may include an implant attachment member which may couple with the pivot arm. The implant attachment member may include a receiving portion which may be configured to removably couple with the brain implant. In various implementations, the implant attachment member may be rotatable or allowed to swivel, such as by a screw or other suitable means, to allow the implant to be rotated.

The receiving member may include one or more protruding pegs, extending outwardly from the implant attachment member and may be dimensioned such that the implant is releasably secured by the pegs. For example, the receiving member may have four pegs which are circumferentially positioned around the edge of the receiving member, creating an opening of the same or similar size as the implant. In operation, the implant may be frictionally secured between the pegs, and may be released from the receiving member by overcoming the friction applied by the pegs. In other implementations, suitable alternatives for the pegs may be provided, such as a circular or semi-circular ridge which similarly applies friction to the edge of the implant.

In another aspect, the implant attachment member may be movably attached to the pivot arm via a lowering assembly. The lowering assembly may include a lowering plate which couples to the implant attachment member. The plate may be vertically movable by one or more screws. For example, a lowering screw may attach the plate to the pivoting arm such that by actuating the screw, the lowering plate is moved, creating a space between the pivot arm and the lowering plate. In operation, while in the second configuration, the actuating (e.g. tightening) of the screw may extend the implant towards (or into) the implant placement site, to be placed in position to be secured to the cranium. The lowering screw may then be retracted such that the plate is pulled away from the implant and towards the pivot arm.

In another aspect, the brain implant assembly may include a mount attachment, which may be removably attached to the base member. The mount attachment may provide a linear or semi linear arm which is configured to span parallel with the cranium near the implant placement site. In various implementations, the mount attachment may include one or more nozzle holders. The nozzle holders may act to receive one or more nozzles which are configured to provide suction and/or saline for the implant placement site. Such nozzles may include a proximal end, such as a threadable opening, to attach to supply tubing, and a distal end, such as a spout which may extend proximally to the implant placement site. In some implementations, a nozzle intended for suction may have a distal end which may extend within the implant placement site, such that the suction nozzle is proximal to the brain. In some aspects, the nozzle holders may be moveable or adjustable, including being moveable vertically, relative to the mount attachment. For example, the nozzle holders may be adjustable vertically such that they can be positioned closer to or farther from the implant placement site.

In another aspect, the brain implant may include a plurality of flexible electrodes which electrically connect with the brain implant by a plurality of wires. For example, the plurality of wires may extend from the embeddable portion of the brain implant, and the plurality of flexible electrodes may be configured to be inserted into the brain of the patient. In some aspects, the pivot axis of the pivot arm may extend through the flexible electrodes.

In another aspect, the apparatus may include a cartridge, which may be removably coupled with the receiving portion of the implant attachment member. The cartridge may be configured to position each of the plurality of flexible electrodes into a linear or semi-linear array, such as to assist in the insertion of each of the plurality of flexible electrodes into the brain of the patient. The cartridge may further include an identification pattern located on an external face of the cartridge. For example, the identification pattern may provide a visual indicia to assist in locating the position of the cartridge to assist in the process of inserting the electrodes into the cranium. In some implementations, the identification pattern may be visible to a computer.

In another aspect, the apparatus may include a cover which may be removably attached to the pivot arm when the pivot arm is in the first configuration. The cover may protect the implant and/or other electronic or fragile elements of the apparatus from inadvertent damage, including during packaging, shipping, and during implanting into the cranium, for example. The cover may be made of a variety of materials, and may for example be transparent or translucent to allow for a user to see through the cover for better positioning of the apparatus on the cranium, or for positioning relative to the implant placement site. In various implementations, the cover may allow access to certain parts of the apparatus while attached to the pivot arm. For example, a user may be able to fasten screws through the attachment holes of the base member while the cover is still removably attached to the pivot arm. The cover may be attached to the pivot arm by various means, including frictionally attaching to the pivot arm based on the dimensions of the cover and pivot arm.

The cover may further include various features which are configured to assist in the positioning and tilting of the implant to better align with the implant placement cite. For example, the cover may include a level positioned on the exterior surface of the cover, such as a bullseye level, which shows the relative tilt of the implant. The cover may be shaped such as to assist in the placement of the apparatus relative to the implant placement site. For example, the cover may have a distal edge which is shaped to align with the edge of the implant placement site, such that the distal edge of the cover aligns with the brain implant when the pivot arm is rotated to the second position.

Implementations of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to the entire specification of this patent, all drawings and each claim.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 is a top perspective view of a brain implant apparatus with a cover according to implementations of the present technology;

FIG. 2 is a top perspective view of the brain implant apparatus of FIG. 1 without the cover in a first configuration;

FIG. 3 is a perspective view of the brain implant apparatus of FIG. 1 without the cartridge and mount attachment and in the first configuration;

FIG. 4 is a perspective view of the brain implant apparatus of FIG. 1 in the second configuration;

FIG. 5 is a top perspective view of the brain implant apparatus of FIG. 1 in the second configuration with the lowering assembly extended;

FIG. 6 is a top perspective view of the brain implant apparatus of FIG. 1 back in the first configuration with the implant positioned within the implant placement site;

FIG. 7 is a top perspective view of the implant attached to the cranium;

FIG. 8 is an exemplary flowchart of a method of use of the brain implant apparatus;

### DETAILED DESCRIPTION OF THE DRAWINGS

Implementations of the present disclosure are related to a brain implantation tool having a base member that fits to the head of an implantation subject, with an apparatus for stabilizing implantation, and methods of use for the same.

Conventional approaches to implanting a brain implant into the cranium suffer from several limitations. For example, a need has existed for an brain implant apparatus which allows for improved maneuverability and control, to assist with ease of installation of the flexible electrodes into the implant placement site. There is further need for a brain implant apparatus which provides a semi-automated structure which promotes movement which is assistive to the implantation process while preventing undesirable excess movement. More specifically, robotic and computer controlled systems are frequently used for assisting in implanting electrodes within the brain. To aid in providing access to the electrodes as well as the embeddable portion of the implant, a pivotable mechanism which allows the implant to be positioned in an inverted or semi-inverted fashion for the electrode installation process and then pivoted to be positioned proximal to the implant placement site is desirable. Such a pivot or rotation provides desirable access and exposure to the embeddable portion of the implant, as well as a desirable working surface in which a cartridge can be placed to assist the electrode implantation process.

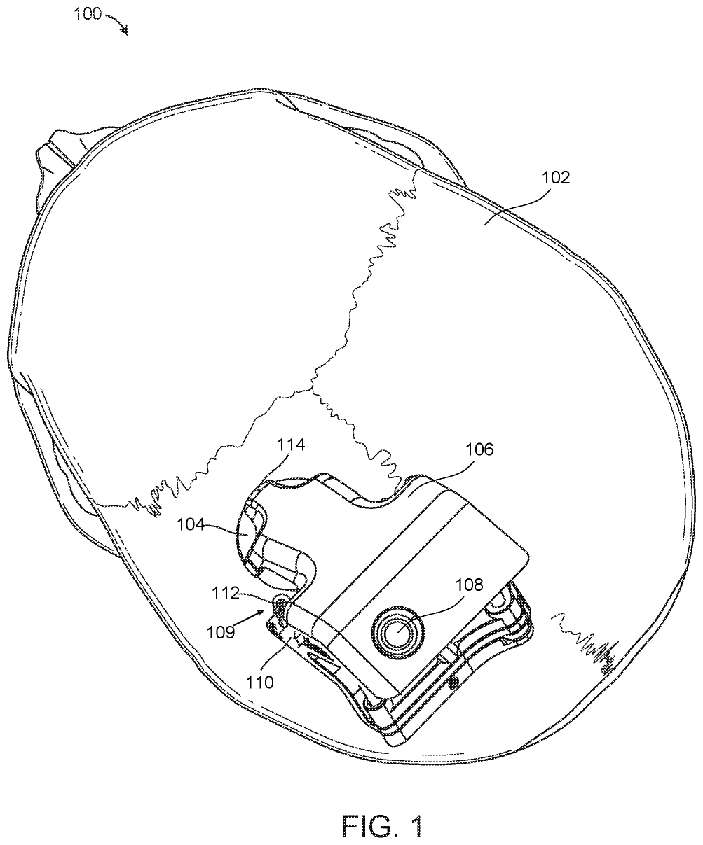

FIG. 1 illustrates a brain implant apparatus 100 intended primarily for placement onto the cranium 102 of a patient or subject proximally to an implant placement site 104. As shown in FIG. 1, the brain implant apparatus 100 includes a cover 106 to protect various aspects of the brain implant apparatus 100 prior to and during initial stages of the installation process. The cover 106 may be transparent such as to assist in the positioning of the brain implant apparatus 100 relative to the implant placement site 104. The cover 106 may further include a level 108, such as a circular or bullseye level, such as to measure and adjust the tilt of the brain implant apparatus 100 relative to the implant placement site 104.

In various implementations, the cover 106 may be dimensioned such that various elements of the brain implant apparatus 100 are exposed when the cover is detached to the remainder of the brain implant apparatus 100. For example, the attachment holes 109 of the base member 110 may be exposed such that apparatus screws 112 can be inserted into the cranium 102 to secure the brain implant apparatus 100, such as with a screwdriver or similar device. Other means of attaching the base member 110 to the cranium 102 are further contemplated other than (or in combination with) apparatus screws 112, such as nails, pins, or other fasters, as may be appreciated by one skilled in the art. Further, the apparatus screws 112 may be biocompatible such that the material and physical properties (e.g. texture) of the apparatus screws 112 prevent or limit negative reactions with the patient when introduced into the cranium 102. Such negative reactions may include, for example, immunological responses within the patient.

The cover 106 may also be dimensioned such that a portion of the cover 106 covers the implant placement site 104 when the brain implant apparatus 100 is installed into the cranium 102. One advantage which may be provided from such dimensioning is to protect the implant placement site 104 from unintended objects for entering the implant placement site 104. The cover 106 being dimensioned to overlap the implant placement site 104 may also provide an alignment advantage. For example, the distal edge 114 of the cover 106 may assist in the placement of the brain implant apparatus 100, such as by the distal edge 114 being dimensioned a specific distance from the base member 110 such that a user knows the base member 110 is correctly positioned for the remainder of the brain implantation process.

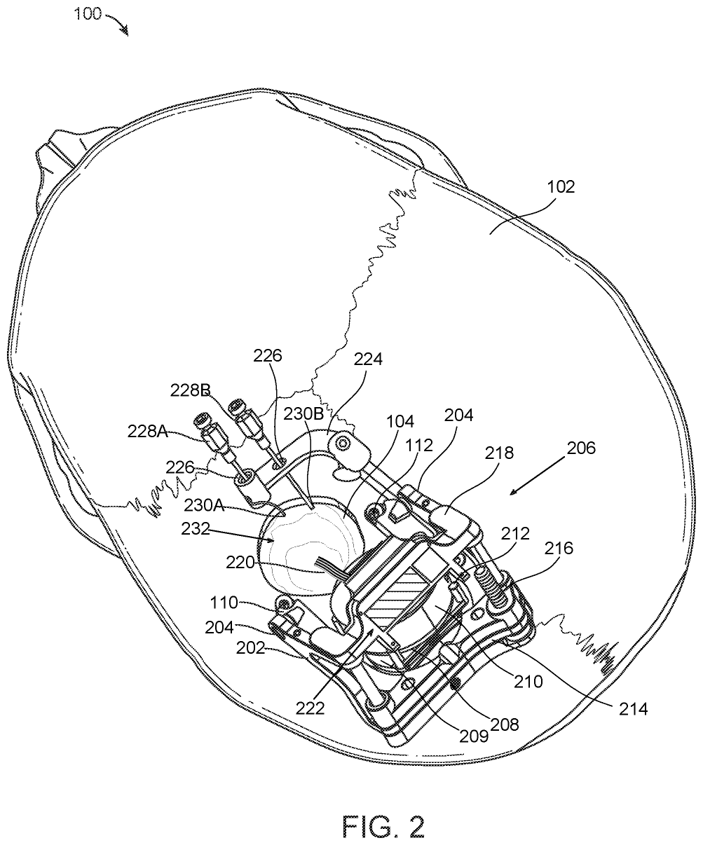

FIG. 2 shows the brain implant apparatus 100 positioned on the cranium 102 of the patient with the cover removed. The brain implant apparatus 100 may further comprise a pivot arm 202 rotatably attached to the base member 110, such as by means of hinge screws 204. As such, the pivot arm 202 is rotatable about a pivot axis which is concentric with the hinge screws 204. As shown in the figure, there are two hinge screws 204 positioned on each side of the base member 110. However, it should be understood that a similar rotation of the pivot arm 202 may be achieved with one screw or with a plurality of screws (e.g. 3 or 4 hinge screws 204. Further, other means can be utilized to provide rotation of the pivot arm 202, such as a swivel joint, swing hinge, or similar mechanism as would be appreciated by one skilled in the art.

As shown in the figure, the pivot arm 202 is positioned in a first configuration 206, such that the pivot arm 202 is positioned substantially above the base member 110. The pivot arm 202 may further include an implant attachment member 208 which may have a receiving portion 209 which releasably attaches to the brain implant 210. In various implementations, the receiving portion 209 of the implant attachment member 208 may include one or more pegs 212 which may frictionally hold the brain implant 210. In various implementations, the implant attachment member 208 may attach to the pivot arm 202 via a lowering assembly 214, which may include a lowering movement screw 216, which is discussed further below.

The brain implant apparatus 100 may further include a cartridge 218, which may be removably attachable to the brain implant 210, such that in the first configuration 206 the implant may be vertically positioned above the brain implant 210 or above other aspects of brain implant apparatus 100. In various implementations, the cartridge 218 may be additionally or alternatively removably attached to the pivot arm 202.

The brain implant 210 may include a plurality of flexible electrodes 220 which may be designed for implantation within the implant placement site 104 of the cranium 102. The flexible electrodes 220 may be in the form of wires from a flexible ribbon cable and attach to an implantable portion of the brain implant 210. The cartridge 218 may assist with the process of inserting the flexible electrodes 220 into the implant placement site 104. For example, prior to insertion within the implant placement site 104, the flexible electrodes 220 may be attached to the cartridge 218 in a position ready to be engaged by a needle and implanted into the implant placement site 104. In various implementations, each of the plurality of flexible electrodes 220 may be positioned on the cartridge 218 in an array such that the needle can more easily retract each of the plurality of flexible electrodes 220 individually. Additional details generally regarding flexible electrodes, and the brain implant 210, for example, may be found in U.S. Patent Publication No. 20180296243, published Oct. 18, 2018, which is hereby incorporated by reference in its entirety.

The cartridge 218 may further include an identification pattern 222 located on the cartridge 218. For example, the identification pattern 222 may provide a visual indicia to assist in locating the position of the cartridge 218 to assist in the process of inserting the plurality of flexible electrodes 220 into the cranium 102. In various implementations, the identification pattern may be visible to a computer, and/or may include a computer-readable pattern, such as a quick response “QR” code, or other barcode. Additional details regarding the cartridge 218 and other features of the present disclosure, for example, may be found in U.S. Pat. No. 11,103,695, issued Aug. 31, 2021, which is hereby incorporated by reference in its entirety.

In various implementations the brain implant apparatus 100 may include a mount attachment 224. The mount attachment 224 may span linearly or semi-linearly near the implant placement site 104, and provide a location to position one or more nozzle holders 226 designed to hold one or more nozzles 228. For example, a suction nozzle 228A may be provided which has a tip 230A which may be positioned inside the implant placement site 104 proximal to the brain 232, such that the nozzle may vacuum any excess fluids from the implantation process. One of the nozzle holders 226 may be designed to hold a saline nozzle 228B to provide saline or other sanitary fluids to assist in cleanliness during the implantation process.

In various implementations, the nozzle holders 226 may be vertically adjustable for better placement of the one or more nozzles 228 relative to the implant placement site 104. For example, the nozzle holders 226 may be moveable or adjustable vertically such that the suction nozzle 228A can be placed in close proximity to the brain 232 of the patient within the implant placement site 104. The saline nozzle 228B may have a shorter tip 230B than the tip 230A of the suction nozzle 228A, as the saline nozzle 228B may not need to be placed in as close of proximity to the brain 232 than the suction nozzle 228A. The one or more nozzles 228 may be configured to receive tubing, which provides the respective saline and suction. To attach the tubing to the one or more nozzles 228, the ends of the nozzles 228 may have threading, barbs, or other suitable means to removably attach the tubing to the nozzles 228.

In various implementations the mount attachment 224 may removably attach to the base member 110. Allowing the mount attachment 224 to be removable from the base member 110 to provide additional space proximal to the implant placement site 104 when the one or more nozzles 228 are no longer needed in the procedure. In various implementations, the mount attachment 224 may additionally or alternatively attach to the cranium 102, such as with biocompatible screws.

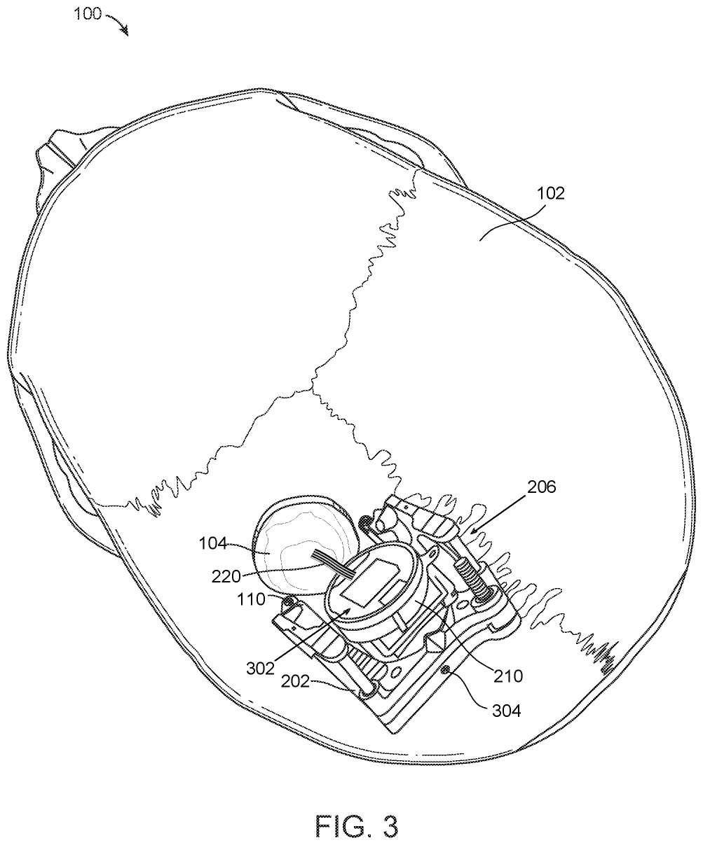

FIG. 3 shows the brain implant apparatus 100 with the mount attachment removed. In addition, the figure shows the brain implant apparatus 100 with the cartridge removed, such that the embeddable portion 302 (the portion ultimately to be received within the implant placement site 104) of the brain implant 210 is clearly visible.

In various implementations, the pivot arm 202 may include a locking screw 304, which acts to secure the pivot arm 202 to the base member 110 in the first configuration 206. As shown in the figure, the locking screw 304 may be inserted through an opening in the pivot arm 202 and thread into the base member 110 to releasably secure the pivot arm 202 to the base member 110. In other implementations, other locations may be suitable to achieve the same function, including a locking screw 304 positioned on the base member 110 itself. Other implementations may also use other means to secure the pivot arm 202 to the base member 110 in the first configuration 206, including a clip, latch, lock, or other suitable means as would be appreciated by one skilled in the art.

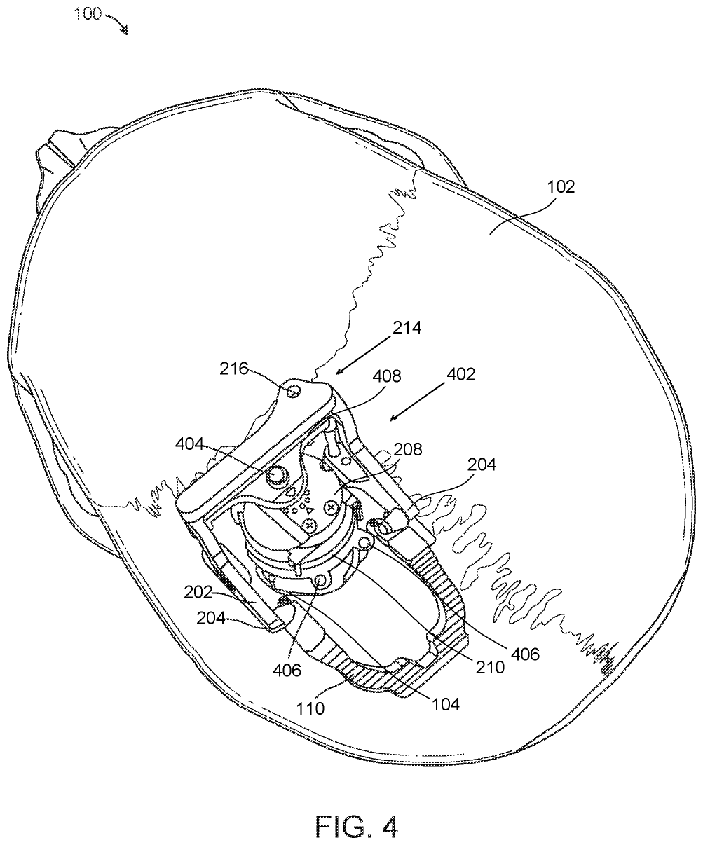

FIG. 4 shows the brain implant apparatus 100. The pivot arm 202 has been rotated about the pivot axis by means of the hinge screws 204 to a second configuration 402. In the second configuration 402, the pivot arm 202 may be positioned such that the brain implant 210 is concentric with the implant placement site 104. In various implementations, the brain implant 210 may be positioned directly above the implant placement site 104.

The implant attachment member 208 may be attached to the pivot arm 202 by a rotating attachment mechanism 404. The rotating attachment mechanism 404 may allow the implant attachment member 208 (and thus the brain implant 210) to rotate. In use, the rotating attachment mechanism 404 may allow the brain implant 210 be rotated while concentric with the implant placement site 104, to assist in positioning the brain implant 210. For example, the brain implant 210 may include one or more mounting eyelets 406 to provide a means to securely attach the brain implant 210 to the exterior surface of the cranium 102. The mounting eyelets 406 may be dimensioned to receive screws, nails, or other suitable means to attach the brain implant 210 to the cranium 102. The rotating attachment mechanism may allow the mounting eyelets 406 to be rotated about the circumference of the implant placement site 104. Such rotation, for example, may allow the mounting eyelets 406 to be placed in a preferred position for ease of attachment to the cranium 102. Specifically, the rotating attachment mechanism 404 may allow for the mounting eyelets 406 to be located such that they can be easily accessed by a screwdriver or other suitable tool.

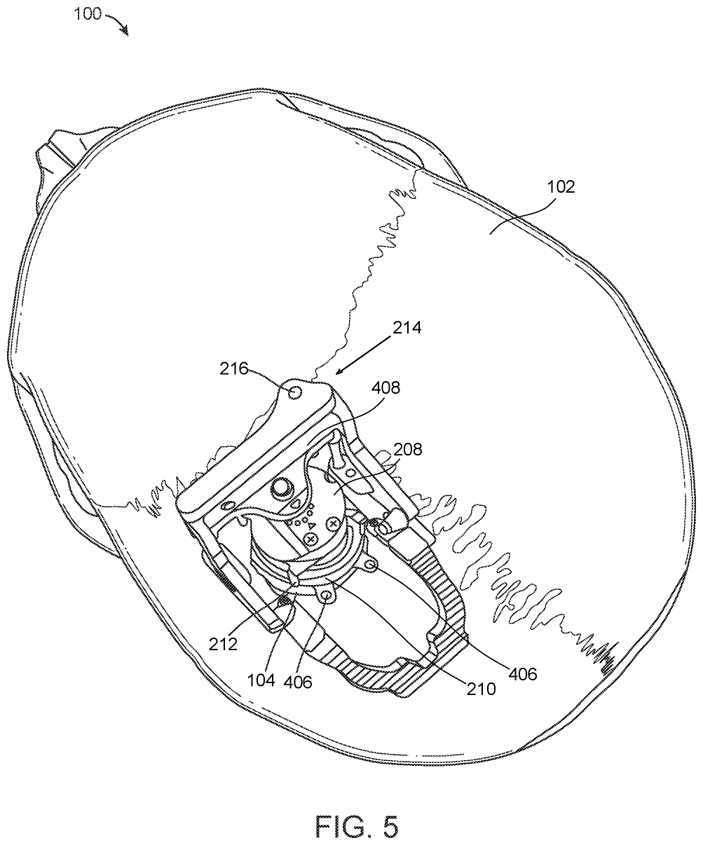

FIG. 5 shows that the lowering assembly 214 allows for the brain implant 210 to be vertically lowered into the implant placement site 104. The lowering assembly 214, may include a lowering plate 408 which is operably attached to the pivot arm 202 by a lowering movement screw 216 (as discussed previously). The lowering movement screw 216 may be actuated to lower the lowering plate 408 away from the remainder of the pivot arm 202 and towards the implant placement site 104.

Referring back to FIG. 4, the brain implant 210 is attached to the implant attachment member 208 and the brain implant 210 is shown positioned vertically above (and concentric with) the implant placement site 104. The embeddable portion 302 of the brain implant 210 may be lowered into the implant placement site 104 by actuating the lowering movement screw 216 to lower the lowering plate 408. The lowering movement screw 216 may be actuated such that the mounting eyelets 406 of the brain implant 210 contact with the outer surface of the cranium 102.

The brain implant 210 may then be detached from the implant attachment member 208. For example, a screwdriver or other tool may be used to overcome the friction between the pegs 212 (see FIG. 5) and the brain implant 210 such that the brain implant 210 is released. Once the brain implant 210 is released, the implant attachment member 208 and lowering plate 408 may be retracted by actuating the lowering movement screw 216 in the reversed direction.

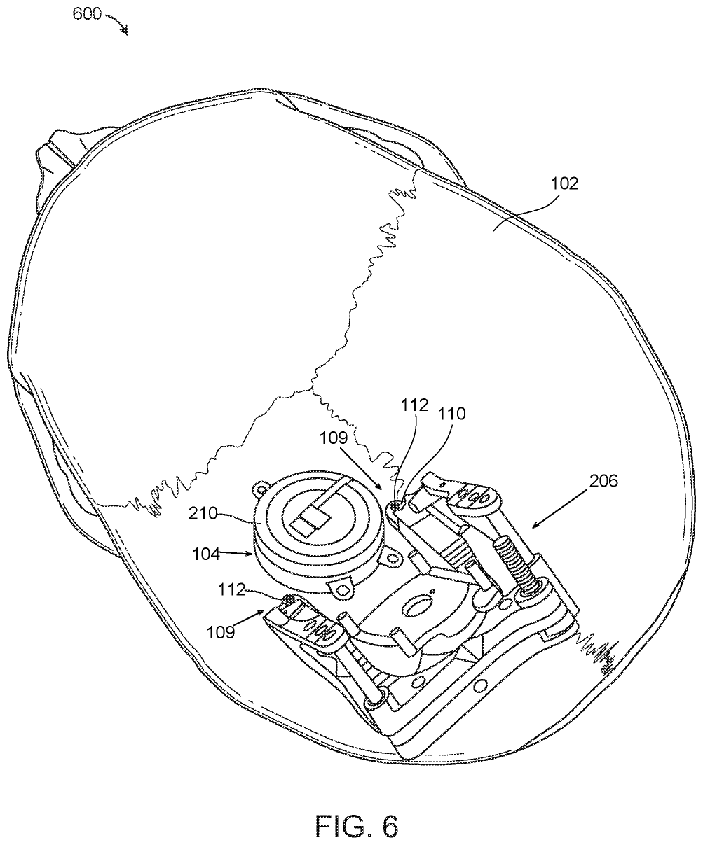

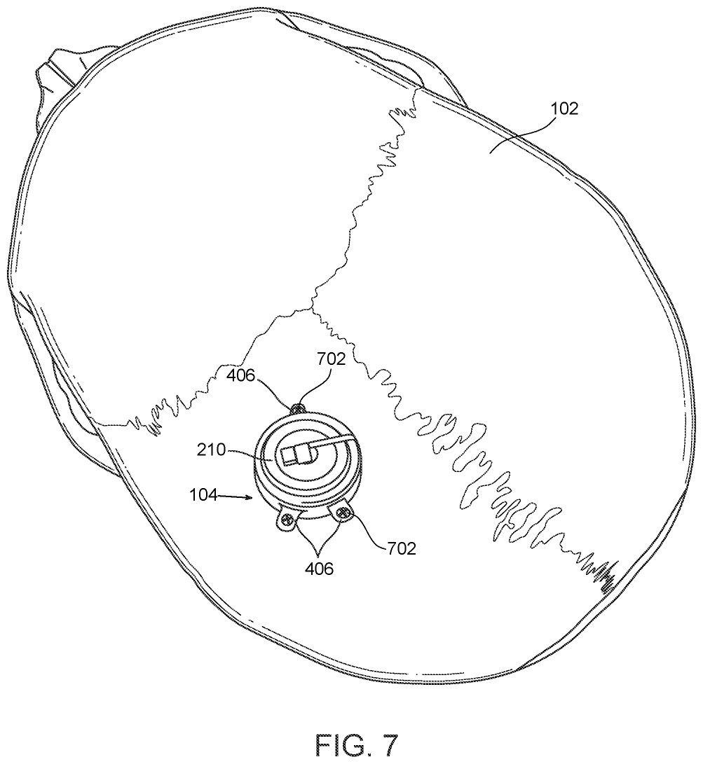

FIG. 6 shows that the brain implant apparatus 100 may be rotated back to the first configuration 206, while the brain implant 210 is maintained within the implant placement site 104. The apparatus screws 112, which removably secured the base member 110 to the cranium 102 may be removed from the attachment holes 109, allowing for the brain implant apparatus 100 to be removed from the cranium 102 (as shown in FIG. 7).

FIG. 7 shows that the brain implant 210 may be secured within the implant placement site 104 by inserting one or more implant attachment screws 702 through the mounting eyelets 406. As discussed with regards to the apparatus screws 112, the implant attachment screws 702 may be biocompatible.

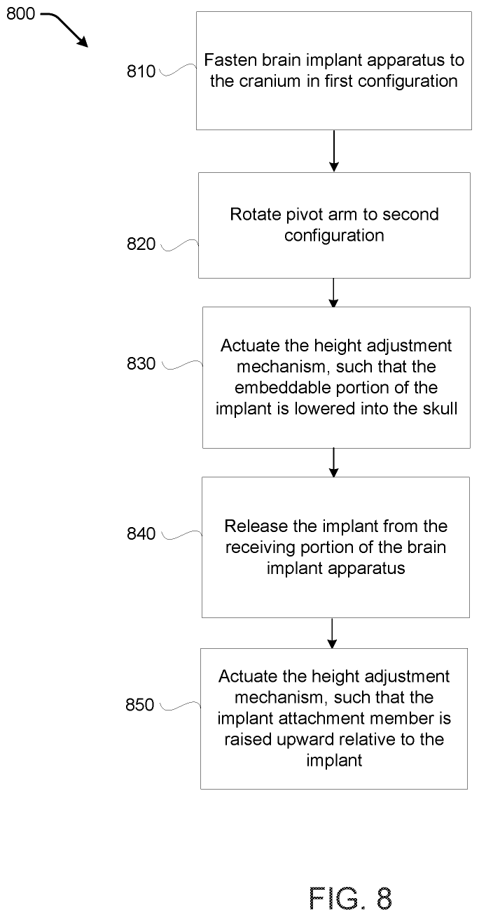

FIG. 8 illustrates a flowchart for an example process 800 for implanting a brain implant 210 within an implant placement site 104 of a cranium 102. In some implementations, one or more process blocks of the figure may be performed by a medical professional (e.g. a doctor, physician, or medical assistant), or other human. In some implementations, one or more process blocks of the figure may be performed by a computer controlled device, including for example an automated or semi-automated, computer controlled device (e.g. a robot).

At block 810, the process 800 may include fastening the brain implant apparatus 100 (see previous FIGS.) to the cranium 102 in the first configuration 206. For example, the base member 110 may be attached to the cranium 102 with screws (e.g. apparatus screws 112), such as biocompatible screws. In the first configuration 206, the brain implant 210 may be facing upwards or substantially upwards, such that the embeddable portion of the brain implant 210 is facing away from the cranium 102. In some implementations, positioning the brain implant apparatus 100 for attachment may include determining both the position on the cranium 102 as well as a tilt angle. The brain implant apparatus 100 may be positioned by using aspects of the brain implant apparatus 100 to determine relative position to the implant placement site 104. For example, the shape of various aspects of the brain implant apparatus 100 may include alignment features to aid in alignment with the implant placement site 104. Further, for example, the brain implant apparatus 100 may include a level, such as a circular or bullseye level (e.g. level 108), to assist in measuring the tilt of the brain implant apparatus 100 in comparison to the implant placement site 104.

In some implementations, the process 800 may include implanting electrodes (e.g. flexible electrodes 220) into the brain 232 within the implant placement site 104 while the pivot arm 202 is in the first configuration. The plurality of electrodes may be operably coupled with the brain implant 210. The implantation process may include positioning a robotic surgical tool above the brain implant apparatus 100. The implant process may further include receiving an electrode of the plurality of electrodes from a cartridge 218 with the robot. The cartridge 218 may be coupled to the embeddable portion 302 of the implant, and/or the cartridge 218 may be positioned relatively above the brain implant 210 while the pivot arm 202 is in the first configuration 206. The process 800 may further include inserting the electrode from the cartridge 218 into the brain 232 within the cranium 102 with the robot. The implantation process may include repeating the steps of receiving the electrode and inserting the electrode from the plurality of electrodes. In some implementations, the implantation process described may be performed after the brain implant apparatus 100 is fastened to the cranium 102. The implantation process may further occur after a cover (e.g. cover 106) is removed from the brain implant apparatus 100.

In some implementations, the process 800 may include positioning a distal end of a suction nozzle 228A proximal to the brain 232 in the implant placement site 104. The suction nozzle 228A may be operably coupled to the base member 110 with a suction nozzle holder (e.g. nozzle holders 226). In some implementations, the position of the suction nozzle holder may be adjustable, such as adjustable vertically, relative to the implant placement site 104.

The process 800 may include activating a vacuum coupled to the suction nozzle 228A by tubing to provide suction through the distal end of the suction nozzle 228A. The vacuum may be configured to remove fluid from within the cranium 102 at the implant placement site 104. The process 800 may include adjusting the height of the distal end of the suction nozzle 228A in the implant placement site 104, such as, for example, such that the distal end is making contact with the brain 232 of the patient, but not providing pressure against the brain 232 (or piercing the brain 232). In addition, the suction nozzle 228A may not work to vacuum material (such as fluids) from the implant placement site 104 if the distal end of the suction nozzle 228A is too distant from the materials and brain 232. The suction nozzle holder may be adjustable such that the optimal or preferred height of the distal end of the suction nozzle 228A is achieved.

In some implementations, the process 800 may include positioning a distal end of a saline nozzle 228B proximal to the cranium 102 in the implant placement site 104. The saline nozzle 228B may be operably coupled to the base member 110 with a saline nozzle holder (e.g. nozzle holders 226). The process 800 may include activating a saline source coupled to the saline nozzle 228B by tubing to provide an outflow of saline through the distal end of the saline nozzle 228B. The saline source may be configured to provide saline within the cranium 102 at the implant placement site 104.

The process 800 may include adjusting the height of the distal end of the saline nozzle 228B in the implant placement site 104, such as, for example, such that the distal end is near enough to the brain 232 of the patient such that the outflow of saline does not excessively splash or splatter when the saline contacts the brain 232, but also not too close to the brain 232 such that the outflow of saline is hindered by contact with the brain 232, or that the pressure of the outflowing saline damages the brain 232 or other tissue.

At block 820, the process 800 can include rotating the pivot arm 202 from the first configuration 206 to a second configuration 402. In the second configuration 402, the embeddable portion 302 of the brain implant 210 is facing downward (or substantially downward) towards the cranium 102 such that the embeddable portion 302 is above an implant placement site 104 in the cranium 102. In some implementations, the embeddable portion 302 is located directly above the implant placement site 104 in the cranium 102. Further, the embeddable portion 302 may be located slightly above the exterior surface of the cranium 102, such that no contact is made between the brain implant 210 and the cranium 102.

In some implementations, the process 800 may include disengaging a lock (e.g. locking screw 304) prior to rotating the pivot arm 202 from the first configuration 206 to the second configuration 402. The lock may be configured to releasably secure the pivot arm 202 to the base member 110 in the first configuration 206. As such, the lock secures the pivot arm 202 to the base member 110 to prevent rotation while the brain implant apparatus 100 is in the first configuration 206. The process of disengaging may include unscrewing a screw, bolt, or similar mechanism. The process of disengaging may, alternatively or in addition, include releasing a latch, hook, clip, or other similar locking structure.

At block 830, the process 800 can include actuating the height adjustment mechanism (e.g. lowering assembly 214), which may be coupled with the pivot arm 202, while the pivot arm 202 is in the second configuration 402, such that the embeddable portion 302 of the brain implant 210 is lowered into the cranium 102 at the implant placement site 104. The actuating may be achieved, for example, by rotating a screw or bolt such that a distance is created between the receiving portion and the pivot arm. Other similar means are contemplated to achieve actuating the function of height adjustment mechanism, including for example, a sliding mechanism which includes a lock to prevent height adjustment when not actuated.

At block 840, the process 800 can include releasing the brain implant 210 from the receiving portion of the brain implant apparatus 100. As described previously, the brain implant 210 may be removably attached to the receiving portion using various means, including for example, friction, magnetics, adhesives, screws, or similar mechanisms as would be appreciated by one skilled in the art. In some implementations, the releasing the brain implant 210 can be achieved by providing pressure between the receiving portion 209 and the brain implant, such that the force securing the implant (friction, magnetic force, etc.) is overcome. For example, a standard screwdriver, or other thin tool may be inserted between the implant and the receiving portion, and force can be applied to pry or dislodge the brain implant 210.

At block 850, the process 800 can include actuating the height adjustment mechanism (e.g. the lowering assembly 214), while the pivot arm is in the second configuration, such that the implant attachment member 208 is raised upward relative to the brain implant 210. The actuating may be achieved, for example, by rotating a screw or bolt (as described above) in the opposite direction from block 830, such that the between the receiving portion 209 and the pivot arm 202 is reduced. Other mechanisms, such as a slide can be actuated, for example, by applying a force in the opposite direction from the block 830.

In some implementations, the process 800 may include securing the brain implant 210 within the implant placement site 104 by fastening a screw (e.g. implant attachment screws 702) through an eyelet (e.g. mounting eyelets 406) of the brain implant 210. The eyelets 406 may provide a means to secure the brain implant 210 to the exterior surface of the cranium 102. The screw or screws may be biocompatible, as described herein. The eyelets 406 may extend radially from the brain implant 210, similar to a tab, and have a hole of suitable size to receive an implant attachment screw 702.

The detailed description of example implementations refers to the accompanying drawings. The same reference numbers in different drawings may identify the same or similar elements. In the following description, for the purpose of explanation, numerous specific details are set forth in order to provide an understanding of various implementations of the subject matter. It will be evident, however, to those skilled in the art, that implementations of the inventive subject matter may be practiced without these specific details. In general, well-known structures and techniques are not necessarily shown in detail.

As will be apparent to those of skill in the art upon reading this disclosure, each of the individual implementations described and illustrated herein has discrete components and features which may be readily separated from or combined with the features of any of the other several implementations without departing from the scope or spirit of the present invention. Any recited method can be carried out in the order of events recited or in any other order that is logically possible.

It is understood that the examples and implementations described herein are for illustrative purposes and that various modifications or changes in light thereof will be suggested to persons skilled in the art and are to be included within the spirit and purview of this application and the scope of the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Although the terms “first”, “second”, etc. may be used herein to describe various elements, components, regions, layers and/or sections, it should be understood that they should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

As used herein, “removably attached,” “removably coupled,” “releasably attached,” or “releasably coupled” may refer to components that are attached and can be detached relatively easily. For example, magnetically attached components, and components snapped together via mechanical attachments that are loosened with a simple motion.

What is claimed is:

1. A brain implant placement apparatus, comprising:
a base member having a bearing surface contoured to rest against a cranium of a patient proximal to an implant placement site;
a pivot arm hingedly coupled to the base member, wherein the pivot arm is configured to rotate from a first configuration to a second configuration;
an implant attachment member coupled to the pivot arm via a rotating attachment mechanism, wherein the implant attachment member comprises a receiving portion configured to removably couple with a brain implant and rotate the brain implant;
wherein in the first configuration the pivot arm is positioned such that the receiving portion of the implant attachment member is facing substantially upwards, away from the cranium; and
wherein in the second configuration the pivot arm is positioned such that the receiving portion of the implant attachment member is facing substantially downwards, toward the cranium.

2. The apparatus of claim 1, further comprising:
a lowering assembly coupled to the implant attachment member, the lowering assembly including a lowering bolt with screw threads, wherein in the second configuration the implant attachment member is moveable toward or away from the cranium.

3. The apparatus of claim 1, further comprising:
a mount attachment removably coupled to the base member, the mount attachment configured to couple to the cranium of the patient, wherein the mount attachment comprises a nozzle holder.

4. The apparatus of claim 3, wherein the nozzle holder is moveable vertically relative to the mount attachment.

5. The apparatus of claim 1, wherein the pivot arm further comprises:
a locking screw, wherein in the first configuration the locking screw fastens the pivot arm to the base member, preventing rotation of the pivot arm.

6. The apparatus of claim 1, further comprising:
a cover removably attached to the pivot arm when the pivot arm is in the first configuration and configured to protect the brain implant.

7. The apparatus of claim 6, wherein the cover further comprises:
a bullseye level configured to measure a tilt of the brain implant.

8. The apparatus of claim 6, wherein the cover is transparent such that the cover is configured to allow for positioning of the base member relative to the implant placement site.

9. The apparatus of claim 1, further comprising:
the brain implant removably coupled with the receiving portion of the implant attachment member;
a plurality of flexible electrodes electrically connected with the brain implant by a plurality of wires; and
a cartridge removably coupled to the brain implant, the cartridge configured to position each of the plurality of electrodes into a linear array.

10. The apparatus of claim 9, wherein a pivot axis of the pivot arm extends through the flexible electrodes.

11. The apparatus of claim 9, wherein the cartridge further comprises:
an identification pattern on an external face of the cartridge, the pattern configured to be scannable by a sensor of a computer and configured to provide the computer with the position of the plurality of electrodes.

12. The apparatus of claim 1, wherein the base member further comprises:
attachment holes for inserting screws to attach the base member to the cranium, the attachment holes being positioned to be accessible while the pivot arm is in the first configuration and the second configuration.

13. The apparatus of claim 12, further comprising:
the screws, dimensioned to be insertable into the attachment holes to secure the base member to the cranium, the screws being biocompatible.

14. A method of placing a brain implant within a cranium, comprising:
providing a brain implant apparatus comprising a base member, a pivot arm hingedly coupled to the base member, a receiving portion operably coupled to the pivot arm, and an implant releasably coupled with the receiving portion of the brain implant apparatus;
fastening the base member to a cranium of a subject while the pivot arm is in a first configuration, wherein in the first configuration the receiving portion holds an embeddable portion of a brain implant facing upward, away from the cranium;
rotating the pivot arm from the first configuration to a second configuration, wherein in the second configuration the embeddable portion of the implant is facing downward toward the cranium such that the embeddable portion is above an implant placement site in the cranium;
actuating a height adjustment mechanism coupled with the pivot arm, while the pivot arm is in the second configuration, such that the embeddable portion of the implant is lowered into the cranium at the implant placement site;
releasing the implant from the receiving portion of the brain implant apparatus; and
actuating the height adjustment mechanism, while the pivot arm is in the second configuration, such that the implant attachment member is raised upward relative to the implant.

15. The method of claim 14, further comprising:
implanting a plurality of electrodes into a brain within the implant placement site while the pivot arm is in the first configuration, wherein the plurality of electrodes are operably coupled with the implant.

16. The method of claim 15, wherein implanting the plurality of electrodes into the brain further comprises:
positioning a robotic surgical tool above the brain implant apparatus;
receiving an electrode of the plurality of electrodes from a cartridge with the robot, the cartridge being coupled to the embeddable portion of the implant and being positioned relatively above the cartridge while the pivot arm is in the first configuration;
inserting the electrode from the cartridge into the brain within the cranium with the robot; and
repeating the steps of receiving the electrode and inserting the electrode for the plurality of electrodes.

17. The method of claim 14, further comprising:
placing the brain implant apparatus on the cranium of the subject while the pivot arm is in the first configuration, the base member having a bearing surface contoured to rest against the cranium; and
adjusting an angle of the brain implant apparatus relative to the cranium with a level coupled to the brain implant apparatus.

18. The method of claim 14, further comprising:
securing the implant within the implant placement site by fastening a biocompatible screw through an eyelet of the implant.

19. The method of claim 14, further comprising:
positioning a distal end of a suction nozzle proximal to the cranium in the implant placement site, the suction nozzle being operably coupled to the base member with a suction nozzle holder; and
activating a vacuum coupled to the suction nozzle by tubing to provide suction through the distal end of the suction nozzle, wherein the vacuum is configured to remove fluid from within the cranium at the implant placement site.

20. The method of claim 19, further comprising:
positioning a distal end of a saline nozzle proximal to the cranium in the implant placement site, the saline nozzle being operably coupled to the base member with a saline nozzle holder; and
activating a saline source coupled to the saline nozzle by tubing to provide an outflow of saline through the distal end of the saline nozzle, wherein the saline source is configured to provide saline within the cranium at the implant placement site.

21. The method of claim 14, further comprising:
disengaging a lock prior to rotating the pivot arm from the first configuration to the second configuration, wherein the lock is configured to releasably secure the pivot arm to the base member in the first configuration.
